(19) United States
(12) Plant Patent Application Publication
(10) Pub. No.: US 2015/0189806 P1
(43) Pub. Date: Jul. 2, 2015

(54) Lantana Plant Named 'MANUEL'S RED'

(50) ;

(71) Applicant: Manuel Morales, Royal Oaks, CA (US)

(72) Inventor: Manuel Morales, Royal Oaks, CA (US)

(21) Appl. No.: 13/999,017

(22) Filed: Dec. 31, 2013

(51) Int. Cl. A01H 5/00

(52) U.S. Cl. PLT/227

(57) ABSTRACT

A new and distinct Lantana cultivar named ‘MANUEL'S RED’ is disclosed, characterized by distinctive variegated foliage and solid red flowers. The new variety is a Lantana, normally produced as an outdoor garden or container plant.

### LATIN NAME OF THE GENUS AND SPECIES

Lantana camara

### VARIETY DENOMINATION

‘MANUEL'S RED’

### BACKGROUND OF THE INVENTION

The new Lantana cultivar is a product of a chance discover by the inventor, Manuel Morales. The new variety was discovered as a naturally occurring single branch mutation of the variety Lantana camara ‘Radiation Improved’, unpatented.

Mr. Morales discovered the new variety in a commercial planting of the parent variety at his nursery in Royal Oaks, Calif. during the Fall of 2010.

Asexual reproduction of the new cultivar ‘MANUEL'S RED’ by vegetative cuttings was first performed at a commercial nursery in December 2010. Subsequently, several generations have been produced, and have shown that the unique features of this cultivar are stable and reproduced true to type in successive generations.

### SUMMARY OF THE INVENTION

The cultivar ‘MANUEL'S RED’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘MANUEL'S RED’ These characteristics in combination distinguish ‘MANUEL'S RED’ as a new and distinct Lantana cultivar:

1. Bright red flower color

2. Semi-upright outwardly spreading plant habit

3. Variegated foliage

### PARENT COMPARISON

Plants of the new cultivar ‘MANUEL'S RED’ are comparable to the parent Lantana camara ‘Radiation Improved’, unpatented. The two Lantana varieties are similar in most horticultural characteristics however, the new variety ‘MANUEL'S RED’ differs in having variegated foliage, whereas ‘Radiation Improved’ has solid green foliage. Flower color of the new variety is typically solid red, whereas ‘Radiation Improved’ varies in flower color.

### COMMERCIAL COMPARISON

Plants of the new cultivar ‘MANUEL'S RED’ are comparable to the variety Lantana camara ‘Dallas Red’, unpatented. The two Lantana varieties are similar in most horticultural characteristics however, the new variety ‘MANUEL'S RED’ differs in having variegated foliage, whereas ‘Dallas Red’ had solid green foliage.

Plants of the new cultivar ‘MANUEL'S RED’ are comparable to the commercial variety Lantana ‘Pink Lemonade’ unpatented. The two Lantana varieties are similar in most horticultural characteristics, including the characteristic of variegated foliage. Plants of the new variety ‘MANUEL'S RED’, however, have red flowers, whereas flowers of ‘Pink Lemonade’ are pink and yellow.

### BRIEF DESCRIPTION OF THE PHOTOGRAPH

FIG. 1: Illustrates in full color a closeup view of foliage of ‘Manuel's Red’.

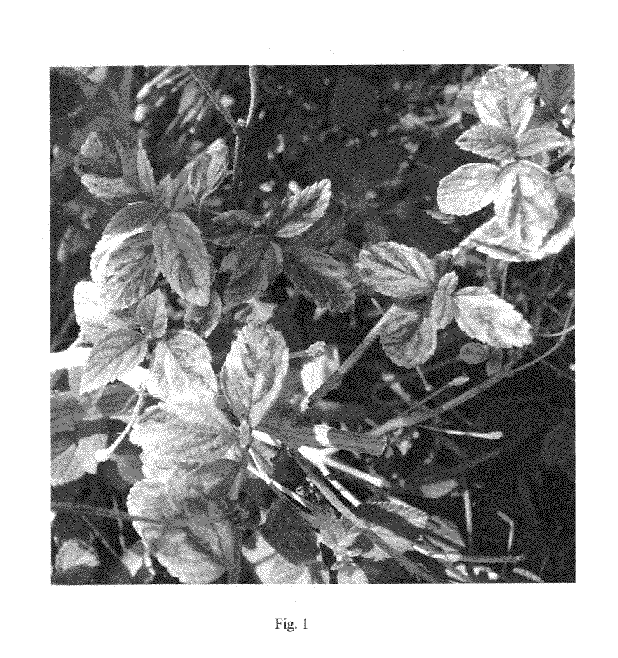

The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘MANUEL'S RED’ plants grown approximately 30 weeks outdoors in Royal Oaks, Calif. The growing temperature ranged from 10° C. to 20° C. during the night and from 15° C. to 35° C. during the day. General light conditions are bright, normal sunlight. Measurements and numerical values represent averages of typical plant types.
Botanical classification: Lantana camara ‘MANUEL'S RED’
Propagation
   i Time to initiate roots.—6 to 8 days at approximately 15° C.
   Root description.—Fibrous.
Plant:
   Growth habit.—Spreading.
   Pot size of plant described.—1 gallon.
   Height.—30 cm.
   Plant spread.—45 cm.
   Growth rate.—Rapid.
   Branching characteristics.—Freely Branches at each internode with two lateral branches.
   Length of primary lateral branches.—Average 33 cm.
   Diameter of lateral branches.—0.7 cm.
   Quantity of primary lateral branches.—4.
   Characteristics of primary lateral branches.—Form: Round Diameter: 0.3 cm. Color: Near Yellow-Green 144B at tip changing to Greyed-Green 197B and 197C Texture: Pubescent, becoming scaly with age Strength: Soft/Very Flexible when young, woody and more brittle with age.
   Internode length.—2 to 4 cm.
Foliage:
   Leaf.—Arrangement: Opposite Quantity: Approximately 14-18 per branch. Average Length: 3.5 cm. Average Width: 1.8 cm. Shape of blade: Ovate Apex: Acute Base: Attenuate Margin: Serrated Texture of top surface: Pubescent Texture of lower surface: Pubescent Aspect: Flat Color: Young foliage upper side: Near RHS Green 137A with Yellow-Green 145B variegation. Young foliage under side: Near RHS Green 137A with Yellow-Green 145B variegation. Mature foliage upper side: Near RHS Green 137A with Yellow-Green 154C variegation. Mature foliage under side: Near RHS Green 137A with Green-Yellow 1B variegation. Venation: Type: Pinnate Venation color upper side: Indistinguishable from leaf blade. Venation color under side: Indistinguishable from leaf blade. Petiole: Length: 0.4 cm. Diameter: 0.15 cm. Color: Near RHS Yellow-Green 144C Texture: Slightly Pubescent.
Flower:
   Natural flowering season.—Continuous Flowers in Warm Climate.
   Days to flowering from rooted cutting.—Approximately 28.
   Inflorescence and flower type and habit.—Corymb.
   Rate of flower opening.—4 to 5 days from bud to fully opened flower.
   Flower longevity on plant.—8-10 days.
   Persistent or self-cleaning.—Self Cleaning.
   Overall inflorescence size.—Height: Approximately 1.0 cm. Diameter: Approximately 3.0 cm.
   Individual flower size.—Height: Approximately 1.1 cm including corolla tube Diameter: Approximately 0.9 cm.
   Bud.—Shape: Squared tubular Length: 1.0 cm Including corolla tube Diameter: 0.3 cm Color: Near RHS Red 45B.
   Corolla.—Petals/lobes: Number: 4 Length: 0.5 cm upper — 0.6 cm lower — 0.5 cm lateral petals Width: 0.5 cm upper — 0.7 cm lower — 0.4 cm lateral petals Shape: Salverform Aspect: Slightly cupped with a long slender tube Margin: Entire Texture: Glabrous to somewhat velvety.
   Color.—When opening: Upper surface: Near RHS Red 45B Lower surface: Near RHS Red 45B Fully opened: Upper surface: Near RHS Red 45B Lower surface: Near RHS Red 45B Throat/tube: Color: Near RHS Red 45B, inner and outer surfaces. Texture: Smooth.
   Calyx/sepals.—Form: 5 Fused Sepals Length: 0.3 cm Diameter: 0.1 cm Sepal shape: 5 Fused Sepals Form a Tube Sepal margin: Fused Sepal texture: Smooth Sepal color: Upper surface: Yellow-Green 144A Lower surface: Yellow-Green 144B.
   Flower bracts/inflorescence bracts.—Appearance: Matte. Length: Approximately 0.7 cm Width: Approximately 0.1 cm. Shape: Narrow deltoid. Aspect: Cupped Apex: Acute Margin: Entire Texture: Upper (inner) and lower (outer) surfaces: Glabrous Color: Upper (inner) and lower (outer) surfaces: Upper — Green 137C-Lower — Green 137C.
   Peduncles.—Length: Average 1.7 cm. Diameter: Approximately 0.1 cm. Angle: about 35 degrees Strength: Strong Texture: Pubescent Color: Yellow-Green 144A Flower Fragrance: Faint
Reproductive organs:
   Stamens.—Number (per flower): 4 Filament length: 0.1 cm Anthers: Shape: Ovate Length: 0.1 cm Color: Brown 200A.
   Pollen.—Color: Near RHS Greyed-Orange 163A Amount: Sparse To None.
   Pistils.—Quantity per flower: 1 Length: 0.4 cm Styles: Length: 0.3 cm Color: Yellow-Green 144B Ovary color: Green 137D Stigma: Shape: Oval Color: Yellow-Green 144A.
Other characteristics:
   Seed.—Very little seed produced Seed diameter: 0.1 cm. Seed Color: Black 202A.
   Fruit.—Drupe Young Fruit: Drupe; Diameter: 0.3 cm. Color: Yellow-Green 146A. Older Fruit: Diameter: 0.2 cm. Color: Black 202A.
   Disease/pest resistance.—Neither resistance nor susceptibility to pathogens and pests common to Lantana have been observed.
   Temperature tolerance.—The new variety tolerates temperatures between 0 to 40° C.

What is claimed is:

1. A new and distinct cultivar of Lantana plant named ‘MANUEL'S RED’ as herein illustrated and described.
